(19) United States
(12) Patent Application Publication
(10) Pub. No.: US 2010/0240087 A1
(43) Pub. Date: Sep. 23, 2010

(54) METHOD AND SYSTEM FOR THE AUTOMATIC DETECTION AND DIAGNOSIS OF A CANCER STEM CELL

(75) Inventors: Swati Bhattacharyya, Plano, TX (US); Supratik Mukhopadhyay, Providence, UT (US); Debashis Bhattacharya, Plano, TX (US)

Correspondence Address: OHLANDT, GREELEY, RUGGIERO & PERLE, LLP, ONE LANDMARK SQUARE, 10TH FLOOR, STAMFORD, CT, 06901, US

(73) Assignee: Simbiosys Biowares, Inc., Dallas, TX (US)

(21) Appl. No.: 12/408,534

(22) Filed: Mar. 20, 2009

(51) Int. Cl. C12Q 1/02; C12M 1/34

(52) U.S. Cl. 435/29; 435/287.1

(57) ABSTRACT

A method for predetermining whether a cancer has a probability to become metastatic or recurrent, having the steps of: obtaining a sample cell population; assaying the sample cell population; detecting the rate of change of the sample cell population's pH over time; and comparing the pH change versus a predetermined pH rate of change. Also provided is a method of treating a patient having the steps of: isolating a cancer stem cell from a patient; culturing the cancer stem cell to produce a pool of descendant cells; culturing cells from the pool of descendant cells in the presence at least one compound among: anti-cancer drugs, myeloablative, chemotherapeutic, and immunotherapeutic agents, and a combination thereof; assaying over time, during the step of culturing, hydrogen ion concentration in the set of cells; and selecting a candidate therapeutic regimen for the patient based on a result of the assaying step.

### BACKGROUND OF THE INVENTION

1. Field of the Invention

The present invention relates to automated detection and/or diagnosis of cancer stem cells in clinical biopsies and the personalized treatment regimens resulting therefrom.

2. Description of the Related Art

Therapeutic advances over the past few decades now allow most cancer patients to fight the disease with an ever-growing arsenal of chemotherapeutic agents. This leads to significant increase in survival time, decreased side effects and improved quality of life. However, most cancer patients are still vulnerable to eventual relapse and are likely to die from the recurrence of the disease. The basic characteristic of cancer cells is that they are fast-growing and highly mutant cells hypothetically produced due to a multi-step neo-Darwinian evolutionary process involving mutation-selection events at the cellular level (Fearon et al., “A Genetic Model for Colorectal Tumorigenesis”, Cell, 61:759-767 (1990); Nowell, “The Clonal Evolution of Tumor Cell Populations”, Science (Washington, D.C.), 194:23-28 (1976)). Conventional cancer therapeutics are cytotoxic drugs (e.g., alkylating agents such as cyclophosphamide, anti-metabolites such as 5-Fluorouracil, plant alkaloids such as vincristine) or irradiation. These methods kill cancer cells by interfering with numerous cellular mechanisms involved in cell growth and DNA replication. Second generation treatments aim to minimize toxicity and are target based like cytotoxic agents that also possess anti-angiogenic activities. The randomized phase III study of adding bevacezumab, an anti-VEGF antibody, to 5-FU, leucovorin, irinotecan (IFL) in metastatic colorectal cancer patients improved tumor response rate, time to tumor progression and overall survival as compared with IFL alone (Hurvitz et al., 2003). Among the emerging therapies immunotherapy is most promising: here monoclonal antibodies attached to therapeutic moieties, such as toxins, radionuclides or chemotherapeutic agents, are administered such that they will selectively bind to the cancer cells and kill them. Immunotherapy is less toxic than conventional therapy. Similarly, experimental gene-therapies aim to correct or replace mutations (Feng et al., “Neoplastic reversion accomplished by high efficiency adenoviral-mediated delivery of an anti-ras ribozyme”, Cancer Res., 55:2024-2028 (1995); Bischoffet et al., “An adenovirus that replicates in p53-deficient human tumor cells”, Science, 274:373-376 (1997)). However, while all of these methods may eradicate the bulk of the tumor mass by destroying the highly proliferative neoplastic cells, it does not kill any clandestine cells that have stem cell like properties and are called “cancer stem cells”. In due time the cancer stem cells will re-grow the tumor mass following standard therapy causing treatment failure and clinical relapse.

However, it should be noted that there is a body of cancer literature not readily accounted for by standard neo-Darwinian mutation-selection models. Namely, there are a host of independent data describing unexpectedly elevated transformation rates observed in certain carcinogen-treated cells not fully accounted for by somatic mutation alone as well as the capability of some highly malignant tumor types to differentiate or even revert to normal under certain conditions-findings also not readily explained by conventional mutation-selection models (Kennedy et al., “Timing of the steps in transformation of C3H 10T1/2 cells by X-irradiation”, Nature (Lond.), 307: 85-86 (1984); Rubin, “Cancer as a Dynamic Developmental Disorder”, Cancer Res., 45: 2935-2942 (1985); Farber et al., “Cellular Adaptation in the Origin and Development of Cancer”, Cancer Res., 51: 2751-2761 (1991)). Accordingly, alternative models to mutation-selection (e.g., invoking a role for potentially reversible non-mutational/epigenetic alterations as effectors of cellular evolution toward increasing malignancy) have been advanced by several investigators in order to explain these and other related phenomena.

For example, as alluded to, while only a minority of mouse prostate cells exposed to methylcholanthrene initially become transformed, the entire population of treated cells give rise to progeny with an increased propensity for transformation at subsequent cell divisions despite removal of the carcinogenic agent. Similarly, treatment of various rodent cells with other types of carcinogens, namely, X-irradiation or retroviral infection, also results in transformation of progeny of initially untransformed cells at an overall rate difficult to reconcile by somatic mutagenesis alone (Farber et al., “Cellular Adaptation in the Origin and Development of Cancer”, Cancer Res., 51: 2751-2761 (1991)). In addition, Rubin has demonstrated that although only a small fraction of growth-constrained NIH3T3 cells form transformed foci, the entire population of these murine fibroblasts gives rise to clones with elevated transformation rates (Rubin, “Cellular epigenetics: Control of the size, shape, and spatial distribution of transformed foci by interactions between the transformed and nontransformed cells”, Proc. Natl. Acad. Sci. USA, 91: 6619-6623 (1994)). In a related manner, while only a minority of in vivo DMBA-treated murine skin cells become transformed, heritable phenotypic alterations are present in the entire basal skin cell layer exposed to this chemical carcinogen thereby corroborating those mentioned in vitro experiments cited in support of a widespread heritable phenotypic effect by certain carcinogens beyond that which can be attributed wholly to their mutagenic effects (Farber et al., “Cellular Adaptation in the Origin and Development of Cancer”, Cancer Res., 51: 2751-2761 (1991)).

It has also been noted, as mentioned, that a variety of cancer cell types can differentiate to varying degrees. For example, human neuroblastoma cells sprout axons and dendrites when grown as explants and murine leukemic cells differentiate into benign granulocytes and macrophages when grown in vitro. Moreover, tritiated thymidine labeling of rodent squamous cell carcinomas and skeletal muscle tumors illustrates that poorly differentiated cells within these tumors can give rise to well-differentiated squamous epithelia and multinucleated myotubes, respectively. In addition, somatic tissues of transplantable mouse teratocarcinomas have been shown to be benign differentiated progeny of a subpopulation of poorly differentiated embryonal carcinoma cells within these particular tumors. Most recently, all-trans-retinoic acid (ATRA) has been found to be efficacious in the treatment of human acute promyelocytic leukemia (APL) by inducing terminal differentiation of malignant leukocytes (Pierce et al. (eds.), “Cancer: a problem of developmental biology”, New Jersey: Prentice Hall Inc. (1978); Degos et al., “All Trans-Retinoic Acid as a Differentiating Agent in the Treatment of Acute Promyelocytic Leukemia”, Blood, 85: 2643-2653 (1995)).

However, the conventional cancer models failed to adequately explain the persistence of neoplasticity. The first conclusive evidence for cancer stem cells was published in 1997 in Nature Medicine by Bonnet and Dick (Bonnet D. and Dick J E. 1997). Human acute myeloid leukemia (AML) is organized as a hierarchy that originates from a primitive hematopoietic cell (Nat Med 3:730-7.) in a AML patient where they demonstrated that in this patient the cancer cells have originated renewable cells with defective morphogenesis.

The phenotype of prostate cancer stem cells is that of a basal cell and cultures derived from cancers, but not benign tissues. Prostate cancer stem cells express a range of prostate cancer-associated RNAs (Lawson, D. A. 2007). The commonly believed hypothesis of human colorectal cancer progression due to accumulation of mutations in tumor suppressor genes and oncogenes leading to highly invasive and migrating tumor cells fails to explain the complex, heterogeneous histology of colorectal tumors or the good differentiation of metastases (Kang, M. K. 2008, Dalbera, P, 2007). The model of migrating CD133-positive population of tumor stem cells, explains these contradictions in the context of the histology of colorectal tumors. These cells trans-differentiate into epithelial cells, which represent the main mass of the colorectal tumors. Moreover, the tumor stem cells are the active component of migration and invasion, thus conferring the malignant phenotype. Taken together, mutations confer to the tumor cells the capability to live outside of their stem cell niche and intestinal compartment. In addition, the trans-differentiation potential of the tumor cell confers plasticity to the tumor and thus contributes to the heterogeneity of colorectal cancers and in acute myeloid leukemia (Hosen, N. 2007).

Previous studies from different laboratories have demonstrated that human gene expression profiles can be used for predictive analysis of metastasis in humans (Prince, M. E. 2007). Initial validation studies used mouse tumors with both high and low metastatic capacity and different genetic background, i.e. resistant and susceptible to metastatic strains. The results showed that metastatic occurrence correlated with genetic background. These results suggested that genetic background can affects chances of human metastasis occurrence along with acquired somatic mutations. Currently, gene chip or real time PCR assays along with mass spectroscopy can detect human metastasis signature gene expression patterns as compared to normal tissue signatures.

Cancer stem cell and breast cancer: in case of breast cancer—the most extensively studied system for cancer stem cells—studies have revealed that breast cancer is not a single disease. Rather, it is a heterogeneous disease composed of distinct subtypes associated with cancer stem cells and acquired clonal genetic alterations which lead to different clinical outcomes (Rapp, U. R. 2008). So not only it is critical to understand this heterogeneity at the beginning of the treatment it is also crucial to follow up the patients as they continue to be on drugs as their inter- and intra-tumoral progression is key for successful therapeutic interventions.

Cell signaling pathways and biomarkers of cancer stem cells: the currently prevailing ideas that set out to explain the process of metastasis are largely based on observations made on the total tumor cell population, and often focus on tumor-intrinsic properties. Several signal transduction networks have been implicated in the regulation of mammary epithelial stem cell self-renewal and maintenance. These signaling networks include those of the Wnt, Notch, TGFO, EGF, FGF, IGF, and most recently, the Hedgehog (Hh) families of secreted ligands (Peacock, C. D. 2007 008). However, we currently know very little about the cellular and molecular mechanisms by which these signaling pathways function to regulate normal epithelial stem/progenitor cells. What is clear is that the regulatory signaling networks thought to control normal stem/progenitor cell self-renewal and maintenance are well-documented to have contributory roles in mammary cancer development and disease progression when misregulated. In mouse model of breast cancer, stem cells of the MMTV-Wnt-1 were sorted by flow cytometry on Thy1, CD24, and CD45. Sorted cells were then injected into recipient background FBV/NJ female syngeneic mice. In 6 of 7 tumors examined, Thy1+CD24+ cancer cells, which constituted approximately 1-4% of tumor cells, were highly enriched for cells capable of regenerating new tumors when compared to cells of the tumor that did not fit this profile (“Not Thy1+CD24+”). Resultant tumors were of similar phenotypic diversity as the original tumor and behaved in a similar manner when passaged. Microarray analysis comparing Thy1+CD24+ tumor cells to “Not Thy1+CD24+” cells identified a list of differentially expressed genes. Orthologs of these differentially expressed genes predicted survival of human breast cancer patients from two different study groups. These studies suggest that there is a cancer stem cell compartment in the MMTV-Wnt-1 murine breast tumor and that there is a clinical utility of this model for the study of cancer stem cells (Cho, R. W. 2).

The ability to identify those individuals at high risk of disseminated disease at the time of clinical manifestation of a primary cancer could have a significant impact on cancer management. However, as these tests are very expensive and time consuming and require a significant amount of tissue which can only be obtained from invasive procedures, one needs a bulk low cost screening device which, with a fraction of a biopsy sample, will be able to tell whether cancer stem cells are associated with the biopsy or not.

Emerging data suggest that initial responses to anti-cancer drug kills the differentiated cancer cells which make up the bulk of the tumor, while leaving the resistant cancer stem cells untouched, leading to relapse. Overcoming intrinsic and acquired resistance of cancer stem/progenitor cells to clinical treatment represents a major challenge in treating and curing the most aggressive and metastatic cancers. However, though cancer stem cell association has been frequently reported in multiple cancer types (e.g., breast cancer, neuroblastoma, AML, etc.), there is no guarantee that all patients of those diseases will have it. It is essential to know whether the patient is harboring any cancer stem cells or cells with multiple mutations inside the biopsy which will eventually give rise to cancer stem cells in the course of treatment. This is because myeloablative procedures are extremely toxic and when applied along with routine chemotherapy it might not be well tolerated by the patient. However, if there is an easy diagnosis available, it might be feasible to reduce the bulk of the tumor through routine anti-cancer drug regimen and to deliver myeloablative drug in succession only in cases of patients with confirmed cancer stem cell infiltration in tumors. This diagnosis will also help the drug discovery groups focused on development of targeted delivery of myeloablative drugs on the tumor itself. The disclosure described herein describes a device that will make it possible to study clinical biopsy samples for cancer stem cell association in individual patients. This diagnosis in turn will lead to personalized treatment regimen for cancer patients reducing their chances of recurrence and metastasis. This will reduce the overall cost of health care via high throughput diagnosis leading to early intervention.

There is no restriction by cancer type whether a cancer will have stem cell association and which one will not. As more and more cancer types are being studied we are finding cancer stem cell association with more cancer types. Cancer stem cells are the small subset of resistant, if not impervious, cells that have the capacity to perpetuate in the presence of most common cancer drugs and treatments (Crocker, A. K. 2007). Even if chemotherapy and radiation kill 99% of the cell population in malignant tumors and the remaining 1% contains cancer stem cells, the tumor is likely to grow back (Blogesklonny, M. V. 2007). Currently, cancer stem cells have been demonstrated to be associated with breast cancer, prostate cancer, melanomas, head and neck cancer, lymphomas (AML and CML) and brain tumors (Hermann, P. C. 2007). As the device described herein is capable of monitoring cells “real time” in vitro, we propose to develop applications for diagnosis of cancer stem cells in tumor biopsy samples. Most important factors identified to date for occurrence of cancer stem cells are genomic instability, micro-environmental changes, and epigenetic changes. Lack of genomic stability, replication errors and external stress as well as direct forms of DNA damage can induce mutations, which leads to origin of the cancer stem cell phenotype (Kenyon, J. 2007). Capturing stem cells at this transition point represents an exciting field of discovery possibly leading to early detection and therapeutic interventions. Most of the research of cancer stem cells has been restricted to identification of key markers (Skipitsin m 2008). Specific studies modeling the tumor induction of specific cells isolated by surface antigens such as CD44 have demonstrated that these cells are not only present in tumors, but that they are the key units in their tumorogenicity. These findings provide useful insight for disease progression, treatment and metastasis. However, the wide variety of proposed markers, and their similarity to endothelial progenitor cells found in angiogenesis, complicates these studies. This disclosure provides a device “free from any dependence on biomarkers” with direct evidence of whether the cancer stem cells are there or not in a specific tumor biopsy samples. This disclosure provides mathematical models for describing growth kinetics for complicated flexible growth models containing 3 and 4 parameters. The disclosure also includes software that integrates data output from existing statistical analysis methods, which is a more useful tool for modeling cell growth. These models utilize standard statistical processes like goodness of fit, mean square error, R², and residual standard deviation for data analysis along with pH parameters in different combinations of controls and drugs of choice. These models provide description of growth behaviors for continuous output in different fields, for instance cell growth kinetics for cancer versus stem cells.

The present disclosure provides the following advantages over the prior art:

No biomarker dependency: the cancer stem cell detection system and methodology of the present disclosure are based on the basic premise of differential growth kinetics, bypassing any requirement of a validated biomarker target whether proteomic or genomic.

High Throughput and Low Cost: the approach of the present disclosure is based on relatively simple pH measurement, and avoids delays and costs typically associated with biomarker-based detection of cancer stem cells

Flexibility in synthesis and deployment of agents: the present disclosure allows flexible accommodation of software monitoring and control agents that can either be synthesized automatically from a declarative specification of the monitoring or control logic, QoS goals and formal models of available devices, services and models, or can be manually generated. All such agents can be automatically deployed across a network of computing, monitoring and control devices, including a network of virtual machines, through the use of declarative specification of the deployment information.

Formal Guarantee: the software system included in this disclosure provides formal guarantee of the correctness of software agents used to arrive at diagnostic decisions, as well as correctness of software agents used for closed loop control of the environment in which biopsies may be cultured, when such agents are automatically synthesized from appropriate declarative specifications.

The present disclosure incorporates heterogeneous physical devices and biological process models: The framework of the present disclosure incorporates expressive yet tractable languages to describe models of complex heterogeneous physio-biological systems comprising cellular processes, drugs applied, electronic sensors, actuators, etc.

Maintaining data provenance: the present disclosure provides a mechanism for certifying the provenance/pedigree of the data exchanged between processes and helps prevent erroneous conclusions resulting from propagation of erroneous data from one process to others.

Reconfigurability: it is possible using the present disclosure to reconfigure a control algorithm at runtime. Such reconfiguration may include, among others, substituting new services/devices for existing ones and can be used to provide new functionalities in response to changing requirements.

### SUMMARY OF THE INVENTION

A method for predetermining whether a cancer has a probability to become metastatic or recurrent by: obtaining a sample cell population; assaying the sample cell population; detecting the rate of change of the sample cell population's pH over time; and comparing the pH change of the sample cell population versus a predetermined pH rate of change.

The present disclosure also provides a method of treating a patient having the steps of: isolating a cancer stem cell from a patient; culturing the cancer stem cell to produce a pool of descendant cells; culturing a set of cells from the pool of descendant cells in the presence of anti-cancer drugs, myeloablative agents, chemotherapeutic agents, immunotherapeutic agents, or a combination thereof; assaying over time, during the step of culturing, a metric of hydrogen ion concentration in the set of cells; and selecting a candidate therapeutic regimen for the patient based on a result of the assaying step.

Also provided is a system having: a control point; a first sensor having a first input and a first output; a directory that contains a representation of the first sensor; a model generator that is in communication with the control point, with the directory and with the first sensor, wherein: the model generator receives an input; the first input of the first sensor is environmental hydrogen ion concentration; and the first sensor is in communication with the control point.

Further provided is a storage medium having encoded thereon in machine-readable format a set of instructions executable on a process, wherein the set of instructions, when executed by the processor, cause the processor to carry out a method having the steps of: synthesizing, by Curry-Howard-style correspondence from a constructive proof, a model that represents a sensor; detecting a rate of change of a cell population's pH over time; comparing the rate of change versus a predetermined rate of pH change; and predicting, based on the step of comparing, a characteristic of a patient from whom the cell population is derived.

### BRIEF DESCRIPTION OF THE DRAWINGS

FIG. 1A is a diagram of steps for developing personalized medicine.

FIG. 1B is a diagram of steps for biopsy processing.

FIG. 1C is a representation of culture conditions of a single cell suspension derived from a biopsy.

FIG. 1D shows the co-relation of pH and cell growth to time for a typical fast growing cell line.

FIG. 2 is a block schematic representation of a culturing diagnosis and monitoring system.

FIG. 3 is a schematic representation of a process or an architecture of a computing system for detection of stem cells in clinical biopsies, and for control of the culture and probe setup, according to the present disclosure.

FIG. 4A is a graph plotting pH versus time for growing MCF7 cells.

FIG. 4B is a graph plotting pH versus time for growing A431 cells.

FIG. 4C is a graph plotting pH versus time for growing MCF7 and A431 cells in co-culture.

FIGS. 5A-5C show comparative regression analysis results for a co-cultured MCF7/A431 cell population.

FIG. 6 is a graph which shows comparative reduction in pH with different number of LA7 cells supplemented to slow growing neoplastic cells over time.

FIG. 7 is a graph which shows comparative change in pH when mammospheres were treated with anti-cancer drugs, alone, or in combination with myeloablative, and cultured for 10 days.

FIG. 8 is a flow diagram of the process for selecting individual candidate therapy according to the present disclosure.

FIG. 9 is a flow diagram of a method for screening therapies against cancer stem cells according to the present disclosure.

FIG. 10 is a block diagram of a system for executing the methods described in the present disclosure.

FIG. 11 is a diagram of cell growth from a normal to a cancer stem cell phenotype.

### DESCRIPTION OF THE PREFERRED EMBODIMENTS

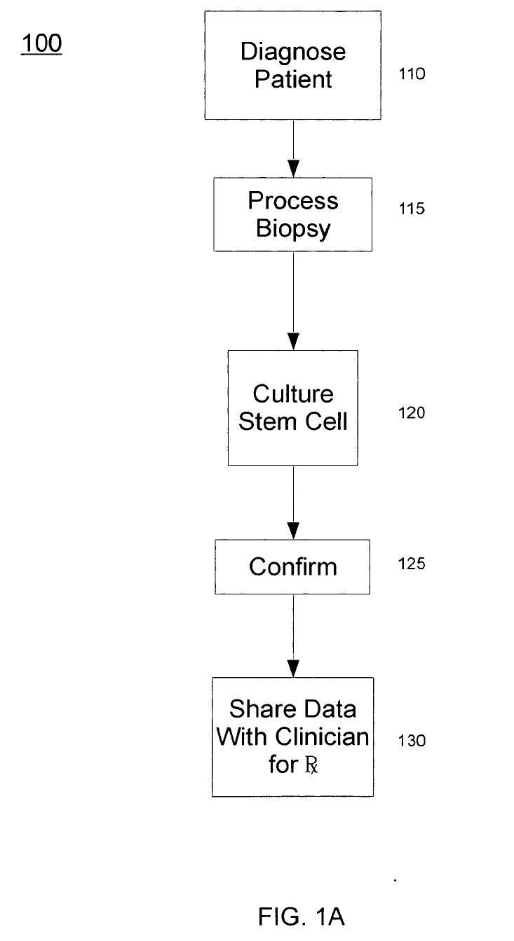

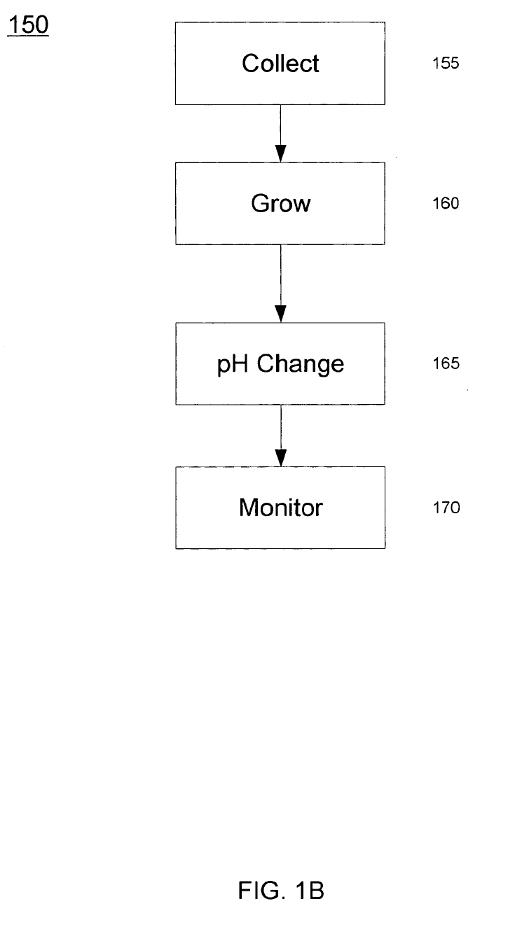

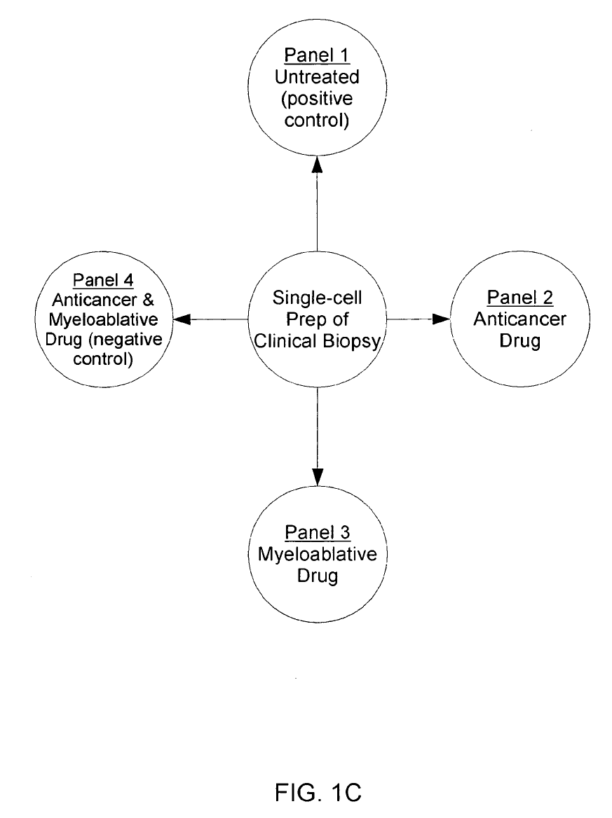

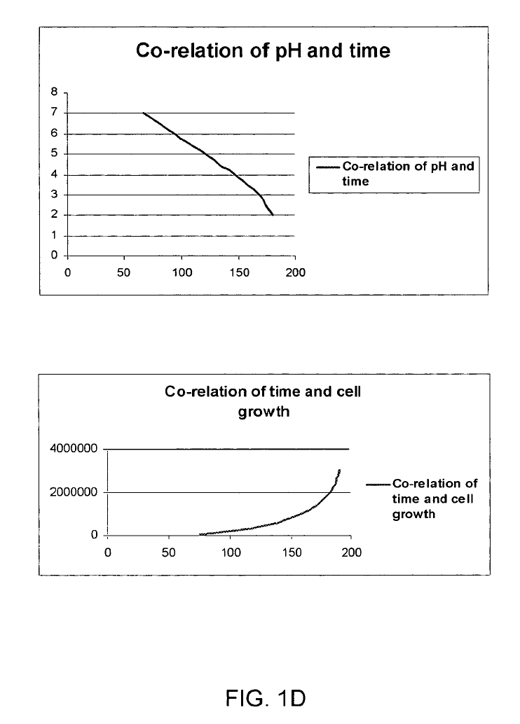

FIG. 1A is a diagram of a method 100 for providing personalized medicine for future oncology treatment, whereby it will be feasible to screen all cancer patients for cancer stem cells in a cost effective manner and in short period of time. At the end of this short period of time, it will be feasible for the clinician using test results obtained according to the present disclosure, to assess individual drug susceptibility and to prescribe a therapeutic regimen customized to the individual patient.

Method 100 begins at step 110. In step 110, a patient is diagnosed with a cancer. It is contemplated that the cancer be of a type in which the involvement of cancer stem cells is known or suspected. It is not necessary that the cancer be of a solid type, thus cancers such as, e.g., Leukemia (CML, AML), Brain, Breast, Colon, Ovary, Pancreas, Prostate, etc., are within the scope of the present disclosure. Method 100 next proceeds to stop 115.

In step 115, a biopsy taken from the patient is processed. Such processing is detailed in the description of FIG. 1B, infra. After processing is completed according to step 115, method 100 next proceeds to step 120.

In step 120, a byproduct of step 115, i.e. a cancer stem cell, is cultured. Step 120 is detailed in the description of FIG. 1C, infra. After culturing is completed according to step 120, method 100 next proceeds to step 125.

In step 125, results of previous steps of method 100 are confirmed for accuracy. The confirmatory tests for cancer stem cells can be done by flow cytometry analysis and sorting of specific population from single cell suspension of tumors followed by real time PCR analysis. The most common antigen studies will be Thy1, CD24, CD34, CD38, CD45, CD44, RT-PCR assays for BMI, Notch 1 and CD133. After confirmation is completed according to step 125, method 100 next proceeds to step 130.

In step 130, a clinician acts on the results of the previous steps of method 100, and decides on a course of treatment that best suits the patient. The clinician may, by way of example, decide to prescribe for the patient a particular chemotherapeutic agent, or combination of therapeutic agents, radiotherapy, immunotherapy, palliative care, or discontinuation of care.

FIG. 1B is a method 150 of biopsy processing. Method 150 begins at step 155. In step 155, a patient biopsy is collected. The biopsy upon collection is validated and graded by a pathologist. This is done through staging, which is a way of describing how much a cancer has grown and spread. A common way of staging cancer is called the TNM classification, in which T stands for tumor (how far a primary tumor has grown locally); N stands for nodes (if the cancer has spread to the local lymph nodes); and M stands for metastases (if the cancer has spread to other parts of the body).

When a cancer is staged, a number is given for each of these three characteristics, 1-4 with 1 being mostly early stage localized conditions and 4 being advanced. In some cases each number may be sub-divided into a, b, c, referring to complicated sub stages of the disease.

Method 150 next proceeds to step 160.

In step 160, the biopsy is homogenized, and a sample of the homogenate is used to seed a culture vessel with cells that are part of the homogenate. The culture vessel is seeded with cells at low cell number; ideally one cell per well. The seeded cells are maintained in conditions compatible with growth. Method 150 next proceeds to step 165.

In step 165, a change in pH takes place in the cell culture vessel according to whether and how vigorously seeded cells grow. Method 150 next proceeds to step 170.

In step 170, a metric of the pH of the culture vessel is measured over time. If the culture vessel's pH changes in a predetermined direction, makes an excursion outside a predetermined boundary, changes at a predetermined rate, or some combination thereof, then the culture vessel is flagged. The typical parameters tracked for quantifying mammalian cell growth kinetics are glucose, dissolved oxygen, lactate and the product of interest. The lactate gets converted to lactic acid and is responsible for the change in pH. For any mammalian cell line if μ is specific growth rate and t_{d} is doubling time t_{d}=ln(2)/μ. Well established methods exist for both intracellular and extracellular methods for pH estimation. FIG. 1D shows the co-relation of pH and cell growth to time for a typical fast growing cell line.

FIG. 1C describes the culture conditions of the single cell suspension derived from the biopsy collected in step 155 of method 150. A clinical biopsy is homogenized, and aliquots containing a single cell or a very small number of cells from the homogenized biopsy are transferred to several culture vessels. A first culture vessel will contain a single cell, and will serve as a positive control (Panel 1). A second culture vessel will contain a single cell and an anticancer drug (Panel 2). A third culture vessel will contain a single cell and a myeloablative drug (Panel 3). A fourth culture vessel will contain a single cell, an anticancer drug, and a myeloablative agent, and will serve as a negative control (Panel 4).

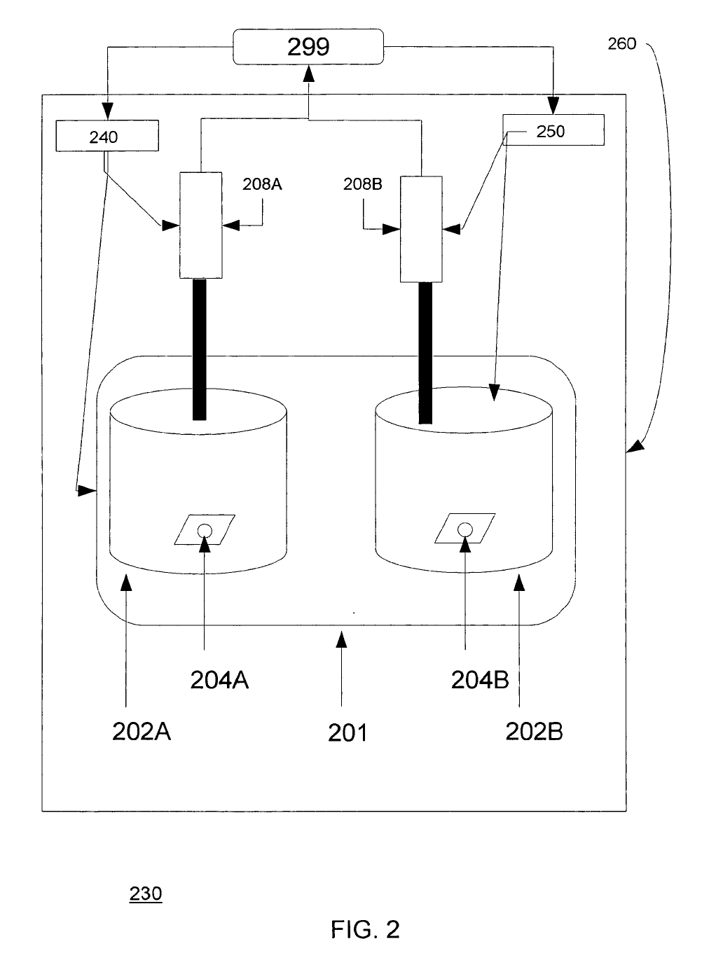

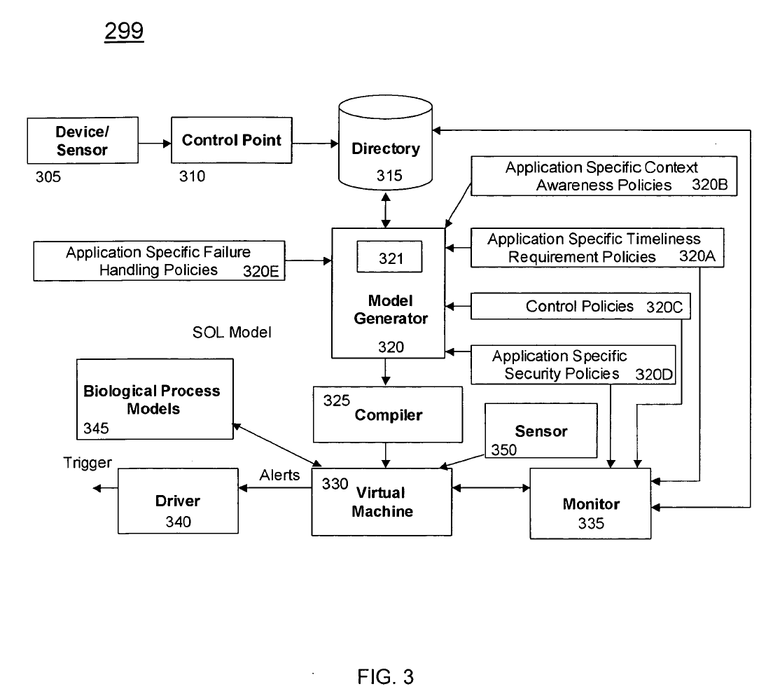

FIG. 2 is a block schematic representation of culturing diagnosis and monitoring system 230, comprising a culture, probe and control device setup 260, and a computing system 299. Computing system 299 is further detailed in FIG. 3. Culture, probe and control device setup 260 in turn provides at least one culture container 201, one or more pH probes such as 208A and 208B, and zero or more control devices 240 and 250. Control device 240 is in communication with pH probe 208A, culture container 201, and computing system 299. Control device 250 is in communication with pH probe 208B, culture vessel 202B, and computing system 299. Culture container 201 in turn provides a plurality of culture vessels, such as culture vessels 202A, 202B.

In each culture vessel 202A, 202B there is grown a population of cells that descend from a progenitor cell. The progenitor cell, e.g. cell 204A, 204B, is selected by dilution from a homogenate prepared from a patient biopsy.

Each culture vessel 202A, 202B is in communication with pH probes 208A and 208B respectively which can employ any known mechanism for pH measurement including electronic, fluorescent, luminescent or radioactive mechanisms, as depicted schematically by 208A, 208B. In one embodiment, each culture vessel of culture container 201 has its own micro pH probe, e.g., micro pH probe 208A. Thus, there could be 96 micro pH probes attached to 96 wells of a microtiter plate.

It is contemplated that culture container 201 and pH probes 208A, 208B and others, will be small enough, along with connectors and fasteners, to be incubated inside a regular CO₂ incubator. However, though it is contemplated that culture container 201 and pH probes 208A and 208B are sized and adapted for use inside a standard CO2 incubator, it is also within the contemplation of the present disclosure that pH probes may suitably be introduced into any in vitro or in vivo environment in which a population of cells of interest may be growing. Changes in pH during cell growth are exemplified herein as due to production of lactate, though any pH-affecting metabolite could be suitably used.

pH probes 208A, 208B are in turn connected to and in communication with a computing system 299 that, among other functions, carries out a function of recording real-time pH data from pH probes 208A, 208B. The real-time pH data are in turn used as input to a pH growth kinetic model.

The pH of a culture vessel, e.g. culture vessel 202A, changes over time as the descendants of progenitor cell 204A metabolize and divide, and is mainly determined by the ionic environment of the of culture vessel 202A (which could be, for example, a microwell). The presence of actively dividing cells effects a decrease in pH. In one embodiment, an alarm sends a lower-bound alert at pH 6. Thus, pH changes are displayed as cell index which has been calibrated against cellular standard curve as described in the above experiment and leads to a relative quantification which can be used to monitor cell viability and number. When there is no cell growth, the pH of culture vessel 202A remains similar to cell culture media alone, and with cell growth the pH of culture vessel 202A will decrease.

For different categories of cancer treatments, it might become necessary to generate a cancer stem cell growth curve over a longer time than the controls and the wells with anti-cancer drug alone. Moreover, long term culture of cells from clinical biopsies will mean exposure to contaminants and detailed SOP has to be developed at phase two for an appropriate surface sterilization step and yet to keep the cell viability intact. Also, this is unlike a control set of experiments where the exact stem cell number is known. Here the total number of stem cells per biopsy will be random. After standardization of the assay with cells, thorough validation will be required along with multiple growth curves with different stem cell count, e.g. from 1-100, and despite that at the end the assay may only be useful qualitatively. Even so it is a low cost realistic and practical solution to a grave problem.

FIG. 3 is a diagram of computing system 299 for detection of stem cells in clinical biopsies, employing a robust distributed computer-based architecture for real-time monitoring of multiple pH probes to diagnose cancer stem cell growth patterns. In some embodiments, computing system 299 can also provide closed-loop feedback directives for control of the culture monitoring and diagnosis system 230.

A networked software system will accumulate and analyze data from multiple implanted sensors (e.g. micro pH probe 208A) for automated monitoring of pH data, possible closed loop control of the cellular environment, and generating intelligent clinical diagnostic conclusions. The data recorded will elucidate growth competition between cancer stem cells and cancerous non stem cells under a set of specifically defined conditions

Computing system 299 provides a control point 310. Control point 310 is in communication with a device/sensor 305, and a directory 315. Device/sensor 305 could be, by way of example, micro pH probe 208A. Control point 310 may be implemented as a general-purpose computer having a processor, a memory, a storage device, and a communication device. Directory 315 is a data structure that contains “specifications” (input thereinto via control point 310) of all available sensors 305, networked computers, medical implants, actuators, and other devices that directory 315 can query. Using standard techniques, directory 315 can be replicated for fault tolerance purposes. The specifications can be provided in the popular UPnP standard (UPnP) and are input through control point 310. Control point 310 can include or be in communication with a compiler, e.g. compiler 325, that converts information into the formal language for delivery to the directory.

Directory 315 in turn is in communication with a model generator 320. Model generator 320 takes as input biological/medical control policies, e.g. control policies 320C, in a “formal” scripting/markup language (obtained by translation from the input provided through graphical user interfaces). The language can express temporal evolution, spatial relationships, communication parameters, departure from and joining of domains protected by firewalls, and/or network topologies. The language incorporates sufficient expressiveness to describe models of complex physical devices (e.g., physical sensors) and services (e.g., online applications) in a heterogeneous network. In addition, the language has constructs for specifying user-specific rules for dealing with uncertainty/noise in the measured data and for managing necessary unit conversions across multiple computing, monitoring and control devices. Model generator 320 also takes as input application-specific policies for handling device/sensor failures, such as application specific failure handling policies 320E (e.g., using redundancy or generating an alert), security, unwarranted situations/contexts, for example application specific context awareness policies 320B, and timeliness, such as application specific real-time constraints 320A, specified in the same “formal” scripting/markup language. In some embodiments, model generator 320 can be configured to generate a proof based on information corresponding to the resources and based on the constraints of the system. For example, model generator 320 can generate a model or constructive proof to determine whether available resources are capable of satisfying the objective of the system. The constructive proof can “contain” instructions for using one or more of the available resources within one or more of the system constraints.

In some embodiments, model generator 320 comprises a deduction engine 321 that can interpret the formal language descriptions as theories, and can syntactically deduce the logical consequences of a set of descriptions. Deduction engine 321 can synthesize a model from the deductions. Synthesis of the models can proceed in any suitable manner. For example, in some embodiments, a so-called Curry-Howard-style correspondence may be used in the synthesis by model generator 320 to synthesize a model from a constructive proof.

The models generated by model generator 320 can be expressed using a modeling language. In some embodiments, the modeling language includes formal operational semantics and incorporates communicating processes with external and internal actions, hierarchical group structure, group communication and logical and physical migration by processes. External actions can involve, for example, communication, logging into and out of groups, etc. Internal actions can involve, for example, invoking APIs provided by the resources. Additionally, the modeling language can communicate time constraints, space constraints, and/or failures, and can include constructs for flow controls. In some arrangements, the modeling language can be dynamically reconfigured, as further discussed below. Such dynamic reconfiguration can involve any suitable replacement method, such as, for example, those used in object oriented paradigms. The modeling language can provide for certification of the provenance of data exchanged via the shell. We will call the models agents.

In some embodiments, the generation of the agents can be completely automatic, which in other embodiments the agents could be manually generated. The agents are compiled to executable code and deployed in accordance with their deployment information on abstract machines running on different hosts of a wired/wireless network. The abstract machines implement the operational semantics of the modeling language. The agents running on different hosts can get input from the different sensors monitoring the cellular processes. They consult reactive models of cellular processes (again agents) running on other hosts of the network (e.g., networked clusters) to determine the current state of the monitored cellular process. The reactive models can incorporate handling of uncertainty, and can use such information to improve accuracy of diagnostic decisions arrived at, given measured pH values which contain significant noise or uncertainty. In some embodiments, the agents can generate control actions that are sent as trigger messages to different actuators connected to the network (e.g., an implanted drug delivery system in a cancer patient, monitored and controlled through a wireless network employing such agents in the implanted system as well as in remote computing devices). Such agents can also monitor every abstract machine to detect any to initiate recovery actions to ensure that a consistent state is reached.

In some embodiments, computing system 299 can automatically deploy intelligent software agents that will run on a reliable infrastructure, “follow” cells in vitro, receive input from probes, e.g., device/sensor 305, and make diagnostic and modulation decisions that will be communicated to, by way of example, appropriate databases, personnel, and devices. In such embodiments, the agents are automatically generated based on a specification in a formal platform-independent scripting/markup language for declaratively specifying medical and control policies as well as cell growth kinetics models of cancer biopsy samples. Control policies, e.g., 320C, can be declaratively specified using an intuitive graphical user interface appropriate for use by a human operator. The specifications are automatically translated to the formal scripting/markup language using standard translation techniques. Distributed intelligent executable “agents” can be automatically synthesized from the translated specifications. The agents use the inputs of the sensors to generate diagnostic decisions. In some embodiments, the agents can use additional specifications provided by the human operator to generate real-time control decisions for managing cellular processes. The fundamental combinators of any appropriate specification language are rooted in mathematical logic (e.g., Blackburn, de Rijke, Venema, 2003) with appropriate combinators added to the language to handle uncertainty.

As an example, one can consider a redox control and sensor system, e.g., computing system 299, for cellular modulation. A sensor, e.g., device/sensor 305, measures the redox potential (E) that is used as an input for a closed loop feedback control system. An actuator controls the amount of gas input based on the measured Eh as well as the control policies. If Eh is too low, lactate production is delayed while hydrogen peroxide production increases. This control policy can be specified as follows:

E=Always(toolow→Eventually(low(lactate) [symbol] high(hydrogen_peroxide)))

where “Always” and “Eventually” are temporal operators. A control policy stating that if controller-sensed E is too low then the controller shall increase the input of gas can be described as

Always(Known(Eh=toolow)→Eventually(action(controller, gas_input, increase)))

where Known is a modality that denotes knowledge of an object or a fact. It is possible to include information about uncertain behavior specifications. For example, there could be user-defined application-specific “atomic” constraints for describing relations among variables, and different modalities (e.g., Eventually, action, etc.) to describe different modes of system dynamics as well as system resources such as available devices and services and their input-output behavior.

Model-Based Monitoring and Control of Cellular Processes

Given the different inputs from sensors monitoring a cellular process, software agents try to determine the possible “current state” at which the cellular process is operating. This state information is used to generate diagnostic decisions. In some embodiments, this state information and pre-defined control policies are used and control actions for modulating the process. A diagnosis system solves a diagnosis problem by comparing a behavior description model with the behavior observed via inputs received from the sensors. If the observed behavior differs from the expected behavior, the diagnosis system generates rank ordered list of possible explanations for such deviation. If a control system comprising one ore more control agents is deployed, this system attempts to issue control actions to revert the deviant state to a normal state. In some embodiments, abduction based diagnosis that utilizes the executable models of the cellular processes and the application-dependent control policies, e.g., control policies 320, can be used to diagnose malfunction and restore normal operation.

### EXAMPLES

### Example 1

It is possible to observe significant difference in the growth kinetics of two different cell lines MCF7 and A431, through measurement of changes in pH. The two cell lines have different doubling time, with A431 being the faster grower with a doubling time of 18 hrs compared to the 27 hrs of the MCF7. In a 96 well plate the cells were seeded in parallel in triplicates with a) MCF 7 b) A431 and c) co-culture of A431 and MCF-7. The cells were seeded at 10K, 5K, 1K, 100, 10 and 1 cell density, respectively, for the single cultures. For the co-culture experiment, 10K MCF 7 cells were seeded as feeder cells and A431 was added as 10K, 5K, 1K, 100, 10 and 1 cell per well in triplicate.

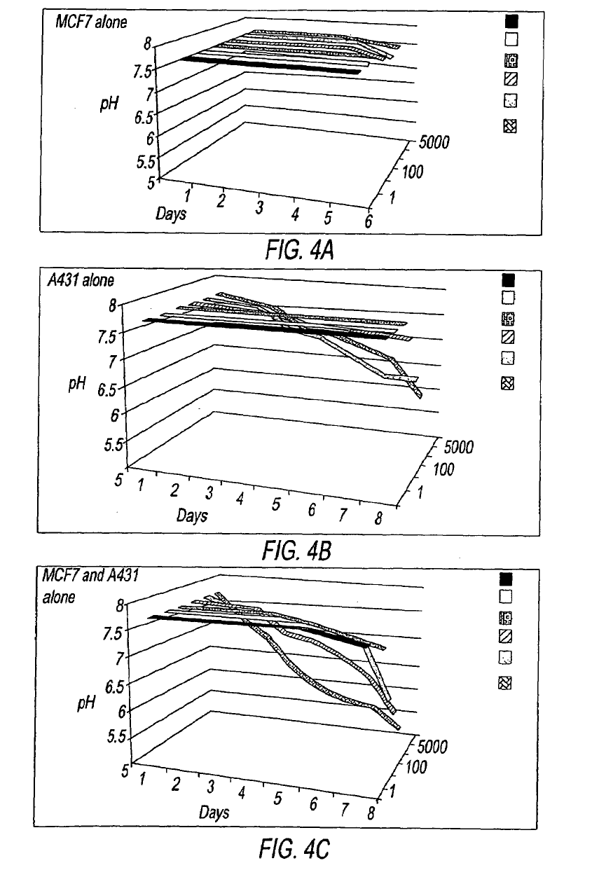

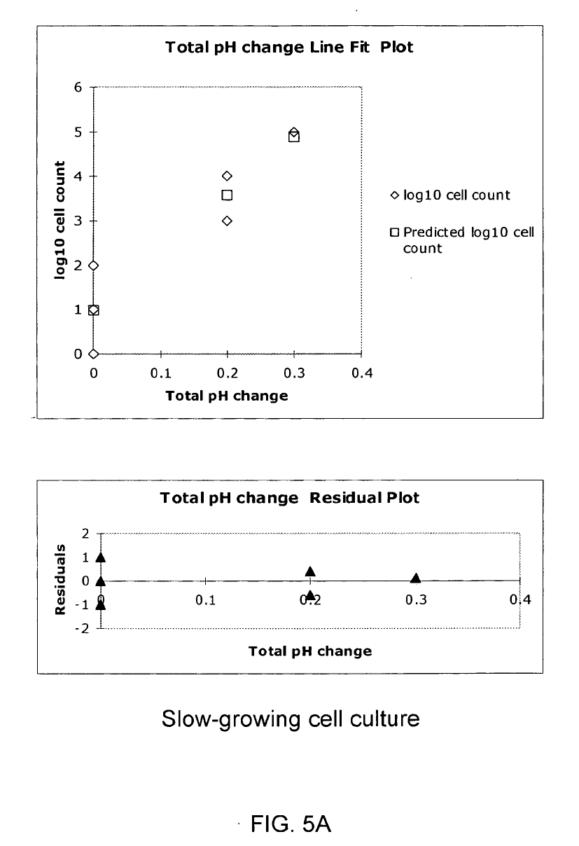

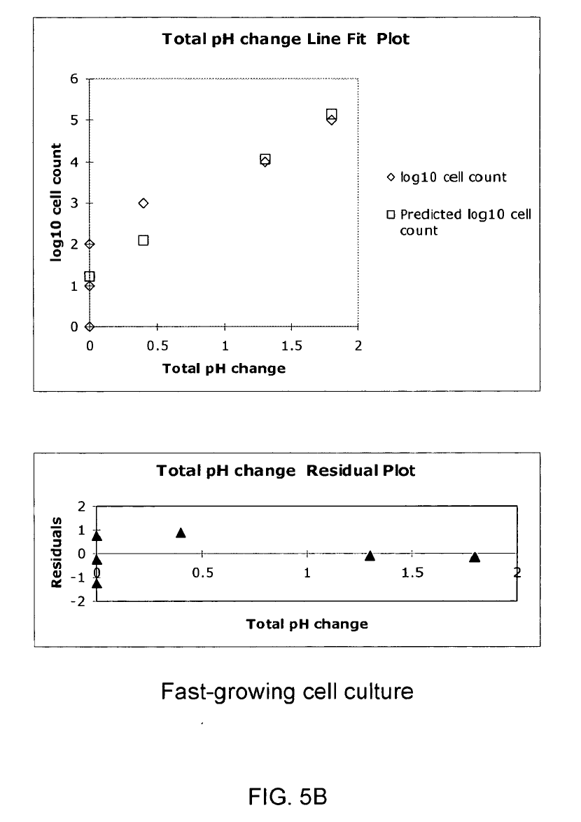

FIG. 4A is a graph plotting pH versus time for growing MCF7 cells for 6 days.

FIG. 4B is a graph plotting pH versus time for growing A431 cells for over 8 days.

FIG. 4C is a graph plotting pH versus time for growing MCF7 and A431 cells in co-culture over 8 days. The graph illustrates that it is possible to detect 1 fast growing cell in a crowd of 10,000 slow growing but neoplastic cells.

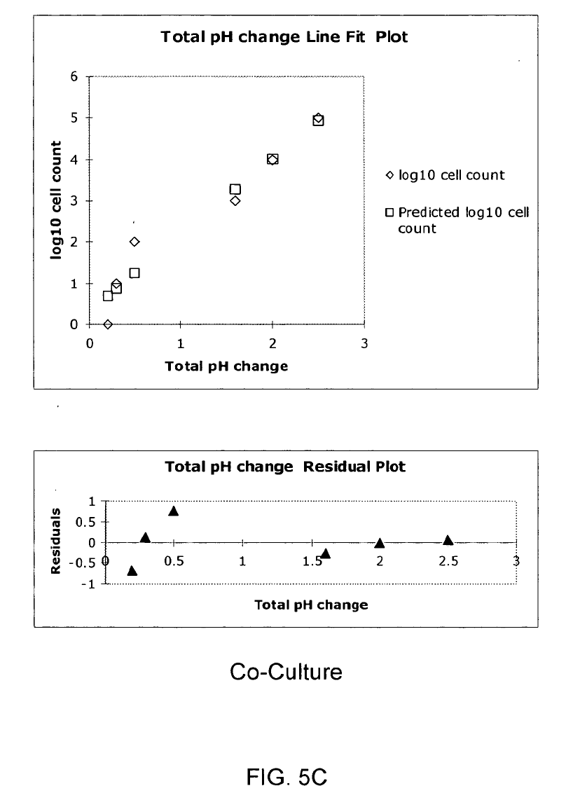

FIGS. 5A-5C show comparative regression analysis results for the same data, using line fit plots and plots of residuals: 5A Slow Growing culture, 5B Fast Growing culture, and 5C Co-culture. Clearly, for the co-culture, the line fit plot is the best, residuals are the smallest, and pH change is most pronounced at low cell counts, demonstrating the power of the present disclosure in detecting a rare cell population in mixed culture of slower and faster growing cells. Other, more sophisticated analysis is clearly possible to yield more sensitive and robust detection capability in a mixed culture of cells.

### Example 2

Experimental design: cell proliferation has been measured by several methods, e.g., MTT, Alamar Blue, lactate dehydrogenase (LDH), fluorimetry, flow cytometry, etc. However, for each of these detection techniques some reagent has to be added to the cells and the culture has to be terminated in order to take a reading. However, all cells while actively growing make the media acidic and in non-metabolic state make the media alkaline. We use these pH changes as a continuous parameter for cell growth kinetics while keeping the cells in culture. The ability of the cancer stem cells to grow faster in vitro and resistance to drugs in vivo are particularly important for isolation of cancer stem cells from biopsies, as there might only be a handful of stem cells in the tumor, and using conventional end point assays it is not possible to detect such a handful. For the assay, we seed 10K, 1K, 100, 10 and 1 LA7 cells in 96 well plates in DMEM Hi glucose media supplemented with B27, 20 ng/ml epidermal growth factor (EGF), 20 ng/ml basic fibroblast growth factor, and 4 μg/ml heparin. We correlate the changes in pH with decreasing cell number and defined a time point when 1 stem cell can make a notable change in pH. LA7 is very aggressive in its mammary cancer stem cell like properties whereby a single LA7 cell can generate mammospheres for greater than 63 passages; co-express lineage specific markers, and generate complex branched duct-like structures and cysts resembling the tubuloalveolar architecture of the mammary tree. Moreover, it has been also reported that the tumorigenic/cancer stem cell behavior of LA7 cells was not due to mutations acquired while establishing the cell lines or from continuous culturing of these cells and lines (Hughes, L. 2008). Therefore, LA7 cells are a model system that can be used to study the cancer stem cell properties associated with tumor formation at the single-cell level. We see faster and steeper reduction in pH due to the cancer stem cell like properties of LA7 and experimental results using LA7 can be used to optimize the device and the computing system, e.g., computing system 299.

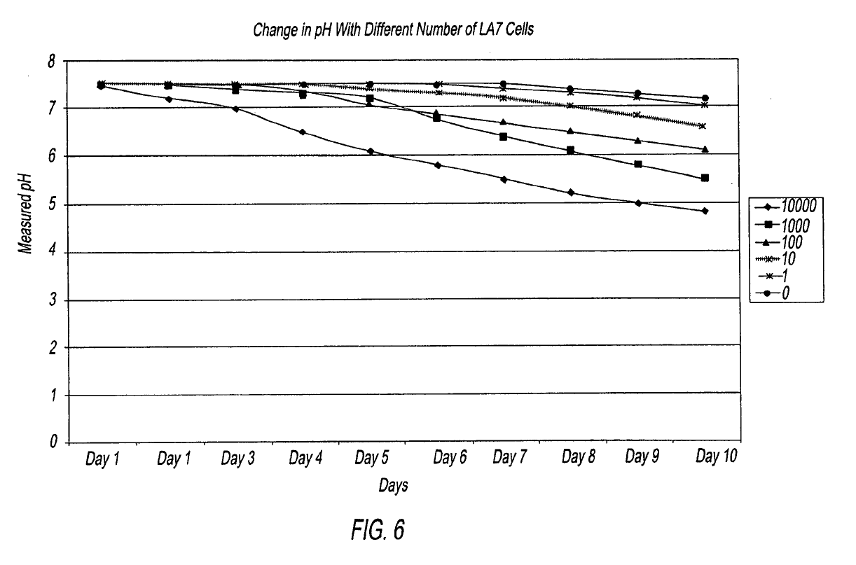

FIG. 6 shows comparative reduction in pH with different number of LA7 cells supplemented to slow growing neoplastic cells.

### Example 3

Experimental design: A single mammosphere is mixed with 10,000 cells of breast cancer cell line MCF-7. These co-cultures run in triplicate, in 4 parallel sets. Panel 1 was the positive control and had no drugs in it, panel 2 received anti-cancer drug cocktail, panel 3 had myeloablative drug in it and panel 4 is the negative control with a combination of anti-cancer and myeloablative drug. We have already shown that it is possible to record a difference in pH after 8 days, when starting with a single cell with a doubling time of 18 hrs. Through supplementation with the single mammosphere, we obtain a model system that can mimic the clinical situation and can be used to study the cancer stem cell properties associated with tumor formation at the single-cell level

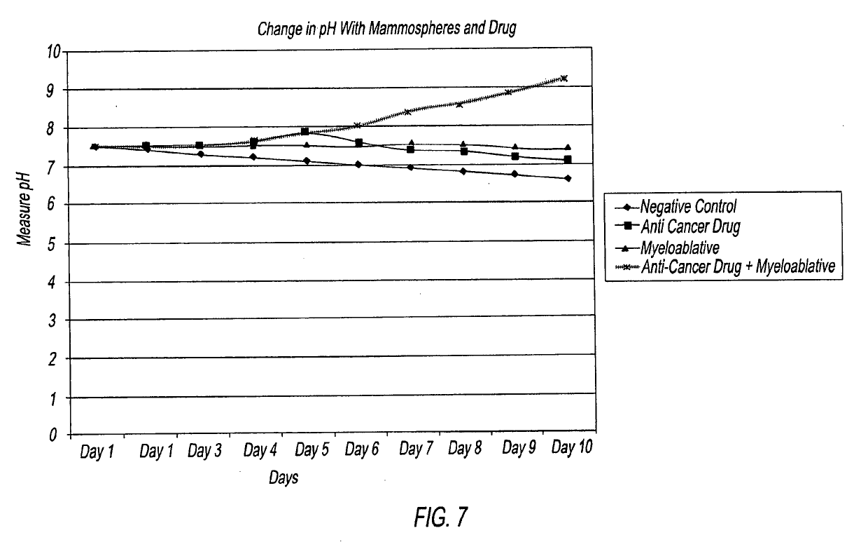

FIG. 7 shows comparative change in pH when mammospheres were treated with anti-cancer drugs, alone, or in combination with a myeloablative agent, and cultured for 10 days.

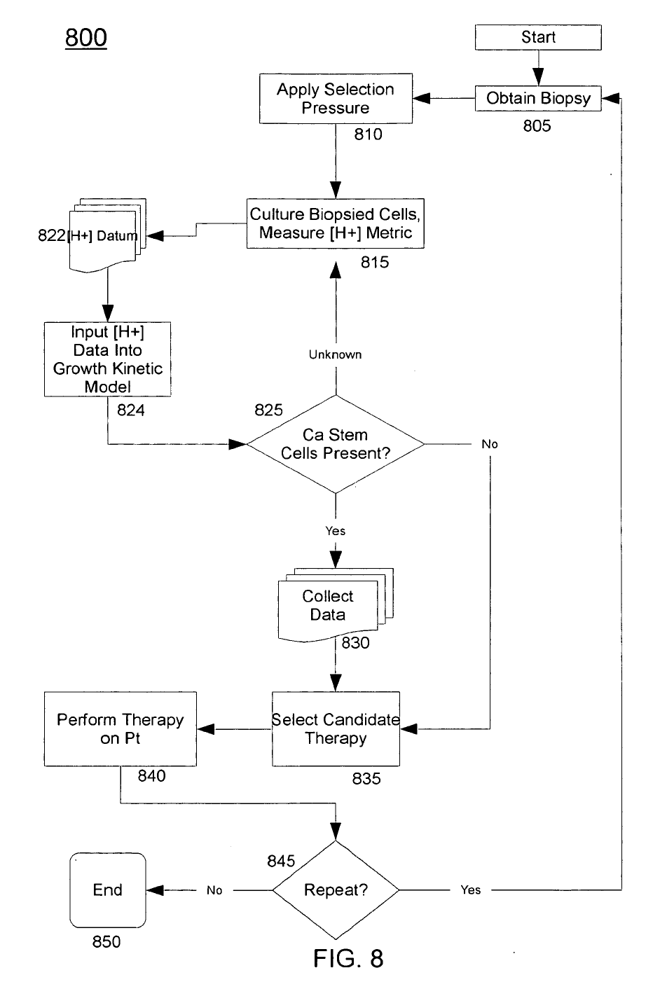

FIG. 8 is a process flow diagram of a process 800 for selecting individual candidate therapy according to the present disclosure. Process 800 begins at step 805.

In step 805, a biopsy or sample is obtained from a patient. It is contemplated that the biopsy be a sample of a cancer. The cancer may be of solid or non-solid type, for example, colorectal cancer or a type of leukemia. The biopsy, especially if from a solid tumor, is prepared for subsequent steps of process 800 by, for example, homogenization, section, digestion, or similar manipulation. Process 800 next proceeds to step 810.

In step 810, a selection pressure is applied to cells of the biopsy taken in step 805. Because step 805 is likely to result in a preparation of cells that contains many cell types (only perhaps one or a few of which are of interest for study) it is necessary to maintain the cells under conditions that promote the growth of cells of interest selectively over cells not of interest. Providing such conditions is referred to herein as applying a selection pressure. Examples of selection pressure to be applied to a prepared biopsy include, without limitation, application of an anticancer agent; application of a myeloablative agent; a combination thereof. Process 800 next proceeds to step 815.

In step 815, the cells of the biopsy are cultured. During such step of culturing, selection pressure may optionally be maintained. Measurement of a metric of proton concentration is undertaken during culture. Process 800 next proceeds to step 822.

In step 822, a proton concentration datum is emitted. Process 800 next proceeds to step 824.

In step 824, the pH datum is input into a cell growth kinetic model.

The growth kinetic model is run, and process 800 next proceeds to step 825.

In step 825 the model outputs whether a cell type of interest, e.g., a cancer stem cell, is present in the culture that emitted the pH datum in step 822. If the growth kinetic model outputs an indeterminate answer, then process 800 returns to step 815. If the growth kinetic model outputs a negative answer, then process 800 proceeds to step 835. If the growth kinetic model outputs a positive answer, then process 800 proceeds to step 830.

In step 830, particular characteristics of the cell type of interest, apart from the cell's mere presence in culture, are aggregated. Process 800 next proceeds to step 835.

In step 835, a candidate therapy for the patient that was the source of the biopsy is selected. It is contemplated that such selection be based on multiple factors, including, without limitation the presence/absence of a cell type of interest, as well as (if present) known or suspected characteristics of the cell type of interest, and known clinical trends for patients having such cells. Process 800 next proceeds to step 840.

In step 840, a clinician performs the candidate therapy on the patient. Process 800 next proceeds to step 845.

In step 845 the clinician decides whether another iteration of process 800 is necessary or desirable for the patient. If yes, then process 800 returns to step 805. If no, then process 800 proceeds to step 850 and terminates.

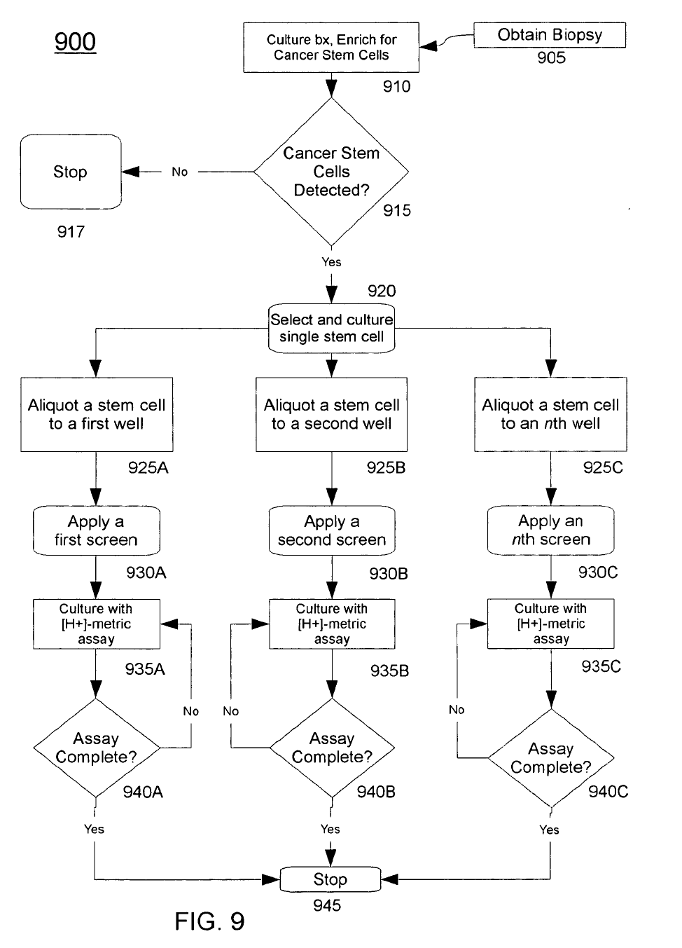

FIG. 9 is a process flow diagram of a method 900 for screening potential therapies against cancer stem cells. Method 900 begins at step 905.

In step 905, a biopsy or sample is obtained from a patient. It is contemplated that the biopsy be a sample of a cancer. The cancer may be of solid or non-solid type, for example colorectal cancer or a type of leukemia. The biopsy, especially if from a solid tumor, is prepared for subsequent steps of method 900 by, for example, homogenization, section, digestion, or similar manipulation. Method 900 next proceeds to step 910.

In step 910, the biopsy is enriched for cancer stem cells. As described supra for method 800, this step of enrichment is effected by application of selection pressure. Method 900 next proceeds to step 915.

In step 915, hydrogen ion concentration data obtained during culture of the biopsy are, as in method 800, used to make a determination whether cancer stem cells are present in the biopsy. If cancer stem cells are not present in the biopsy, then method 900 terminates at step 917. If cancer stem cells are present, then method 900 proceeds to step 920.

In step 920, a single cancer stem cell is selected, e.g. by dilution, from the cultured biopsy. The cancer stem cell is, in turn, cultured, and descendants thereof are aliquotted into first-nth wells of a culture container in steps 925A-C. Further steps of method 900 carried out from steps 925A-C are substantially in parallel, and thus, for clarity, only one path to endpoint will be herein described. Method 900 next proceeds to step 930A.

In step 930A, a first screen is applied to a culture containing a stem cell that is a descendant of the single stem cell of step 920. A screen is, for example, a single chemotherapeutic agent, a combination of chemotherapeutic agents, or indeed any agent having a therapeutic effect to be assayed with respect to a cell of interest. Method 900 next proceeds to step 935A.

In step 935A, the culture of step 930A is maintained over time, either in the presence or absence of selection pressure, as appropriate, and hydrogen ion concentration data are collected over time. The hydrogen ion concentration data are, as with method 800, inputted into a growth kinetic model. Method 900 next proceeds to step 940A.

In step 940A, a clinician determines whether application of a particular screen has had a measurable or desired effect on the cells to which the screen has been applied. If no, i.e. if the assay is not complete, method 900 returns to step 935A. If yes, i.e. if the assay is complete, method terminates at step 945. The clinician will compare the results of steps 925A-940A with, e.g., steps 925B-940B, and so on, in order to select a candidate therapy.

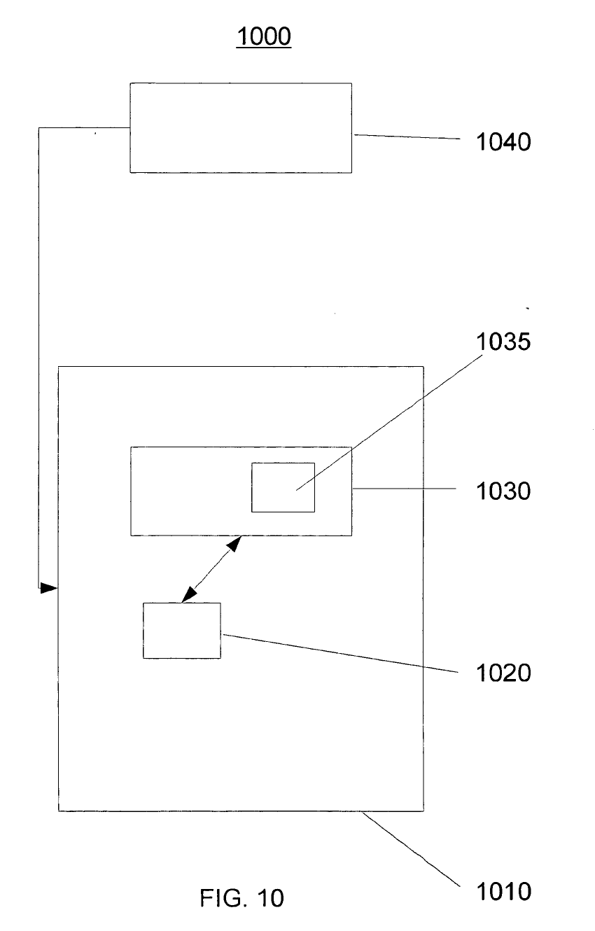

FIG. 10 is a block diagram of a system 1000 for executing the methods described in the present disclosure. System 1000 may be implemented on a general-purpose computer 1010. Although system 1000 is represented herein as a standalone system, it is not limited to such, and may be coupled to other computer systems (not shown) via a suitable communication network (not shown).

Computer 1010 in turn provides a processor 1020 and a memory 1030. Processor 1020 and memory 1030 are in communication. Processor 1020 can be a special-purpose processor or circuitry.

Memory 1030 is a memory that stores data and instructions that control processor 1020. An implementation of memory 1030 would include a random access memory (RAM), a non-volatile data store, or a ROM. One of the components of memory 1030 is a software program 1035.

Program 1035 includes instructions for controlling processor 1020 to execute the processes described in the present disclosure. Program 1035 may be implemented as a single module or as a plurality of modules that operate in cooperation with one another.

The term “module” is used herein to denote a functional operation that may be embodied either as a stand-alone component or as an integrated configuration of a plurality of sub-ordinate components.

Steps associated with these processes can be performed in any order, unless otherwise specified or dictated by the steps themselves.

Although system 1000 is described herein as having the instructions for the processes of the present disclosure installed into memory 1030, the instructions can be tangibly embodied on an external computer-readable storage medium 1040 for subsequent loading into memory 1030. Storage medium 1040 can be any conventional storage medium, including, but not limited to, a floppy disk, a compact disk, a magnetic tape, a read only memory, or an optical storage media. The instructions could also be embodied in a random access memory, or other type of electronic storage, located on a remote storage system and coupled to memory 1030.

Moreover, although program 1035 is described herein as being installed in memory 1030, and therefore being implemented in software, program 1035 could be implemented in any of hardware, firmware, software, or a combination thereof.

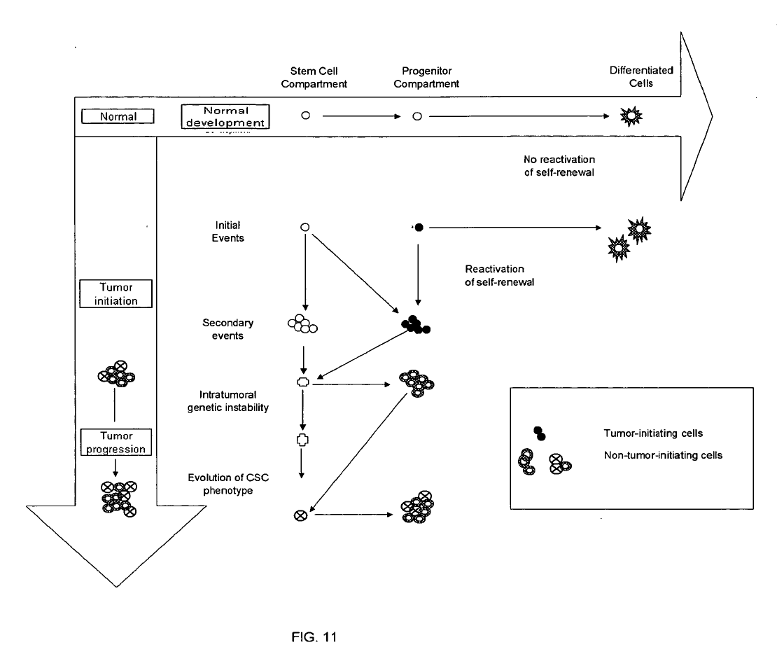

FIG. 11 is a diagram of cell growth from a normal to a cancer stem cell phenotype. In the normal state of development, a cell progresses from the stem cell compartment to the progenitor compartment to differentiated cells.

A normal cell in the progenitor compartment may, in turn, abnormally undergo a reactivation of self-renewal. If such a reactivation takes place, the cell can enter a pathway leading to tumor progression and the evolution of the CSC phenotype. If no such reactivation takes place, the cell proceeds normally to become a differentiated cell.

In tumor initiation, initial events and possibly secondary events (e.g., mutations) take place in a cell in the normal stem cell compartment (or, as described above, in the progenitor compartment). The initial and possibly secondary events in turn are, or introduce, genomic instability into the cell. Thus if the cell is, for example, part of a solid tumor, then the solid tumor is said to have intratumoral genomic instability. Non-solid tumors, such as leukaemias, can similarly be said to experience genomic instability.

A further event can befall the genomically unstable cell. The further event can turn the genomically unstable cell into a non-tumor-initiating cell, or can lead to the evolution of the CSC phenotype in the stem cell compartment. Even such non-tumor-initiating cells can experience an event that transforms them into a tumor-initiating cell in the stem cell compartment.

Tumor-initiating cells of the CSC phenotype, though near an endpoint of the progression from the normal phenotype through to tumor progression, may experience an event that transforms them into non-tumor-initiating cells.

The techniques described herein are exemplary, and should not be construed as implying any particular limitation on the present invention. It should be understood that various alternatives, combinations and modifications could be devised by those skilled in the art. The present invention is intended to embrace all such alternatives, modifications and variances that fall within the scope of the appended claims.

What is claimed is:

1. A method for predetermining whether a cancer has a probability to become metastatic or recurrent, said method comprising:
obtaining a sample cell population;
assaying said sample cell population;
detecting the rate of change of said cell population's pH over time; and
comparing said pH change of said sample cell population versus a predetermined pH rate of change.

2. The method of claim 1, further comprising:
treating said sample cell population to produce an enriched sample cell population;
wherein said sample cell population is a biopsy of a cancer; and said step of treating comprises culturing said sample cell population in the presence of at least one compound selected from the group consisting of: (a) anti-cancer drugs, (b) myeloablative agents, (c) chemotherapeutic agents, (d) immunotherapeutic agents and (e) a combination thereof.

3. The method of claim 1 further comprising:
predicting, based on said step of comparing, a characteristic of a patient from whom said sample cell population was obtained, wherein said characteristic is at least one selected from the group consisting of: (a) the patient's tumor burden; (b) the patient's relapse likelihood; (c) the prevalence of cancer stem cells in the patient, (d) an optimal treatment regimen for the patient; and (e) any combination thereof.

4. The method of claim 2, wherein said cancer is at least one selected from the group consisting of: (a) breast cancer; (b) colorectal cancer; (c) cancer of the head and neck; (d) acute myelogenous leukemia; (d) pancreatic cancer; (e) prostate cancer; (f) neuroblastoma; (g) a cancer of a type in which involvement of cancer stem cells is suspected, known, or sought; and (h) any combination thereof.

5. The method of claim 3, further comprising:
subjecting the patient to a course of treatment that is selected based at least in part on said step of predicting.

6. The method of claim 5, wherein said course of treatment is at least one selected from the group consisting of: (a) continuation of a course of treatment; (b) radiotherapy; (c) immunotherapy; (d) chemotherapy; (e) hormonal therapy; (f) gene therapy; and (g) any combination thereof.

7. A method of treating a patient comprising:
isolating a cancer stem cell from said patient;
culturing said cancer stem cell to produce a pool of descendant cells;
culturing a set of cells from said pool of descendant cells in the presence of at least one compound selected from the group consisting of: anti-cancer drugs, myeloablative agents, chemotherapeutic agents, immunotherapeutic agents, and a combination thereof;
assaying over time, during said step of culturing, a metric of hydrogen ion concentration in said set of cells; and
selecting a candidate therapeutic regimen for said patient based on a result of said assaying step.

8. A system comprising:
a control point;
a sensor, having an input and an output, that is in communication with said control point;
a directory that contains a representation of said sensor; and
a model generator that is in communication with said control point, directory, and sensor, wherein said model generator receives an input from said sensor that is a metric of hydrogen ion concentration.

9. The system of claim 8, further comprising:
a cell culture chamber that is in fluid communication with said sensor.

10. The system of claim 8 wherein said model generator generates a model that represents said sensor, and said model is synthesized by Curry-Howard-style correspondence from a constructive proof, and is expressed in a modeling language.

11. The system of claim 10 wherein said modeling language includes formal operational semantics and incorporates communicating processes with external and internal actions, hierarchical group structure, group communication, and logical and physical migration by processes.

12. A storage medium having encoded thereon in machine-readable format a set of instructions executable on a processor, wherein said set of instructions, when executed by said processor, cause said processor to carry out a method comprising:
detecting a rate of change of a cell population's pH over time;
comparing said rate of change versus a predetermined rate of pH change; and
predicting, based on said step of comparing, a characteristic of a patient from whom said cell population is derived.

13. The storage medium of claim 12 wherein said instructions, when executed by said processor, cause said processor to carry out a method comprising:
synthesizing, by Curry-Howard-style correspondence from a constructive proof, a model that represents a sensor.
